(19) United States
(12) Patent Application Publication
(10) Pub. No.: US 2026/0338969 A1
(43) Pub. Date: Oct. 29, 2026

(54) IMAGING APPARATUS AND METHODS WITH STACKED WIRING LAYERS AND STACKED FILMSBASED IMAGE INTENSIFICATION AND LIGHT RECYCLING

(71) Applicant: REAL TIME IMAGING TECHNOLOGIES, LLC, Charlotte, NC (US)

(72) Inventor: Daniel UZBELGER FELDMAN, Charlotte, NC (US)

(21) Appl. No.: 19/732,562

(22) Filed: Jul. 7, 2026

Related U.S. Application Data
(63) Continuation of application No. 19/158,846, filed on Feb. 22, 2024.
Application No. 19/158,846 is a continuation-in-part of application No. 18/380,530, filed on Oct. 16, 2023.
(60) Provisional application No. 63/486,474, filed on Feb. 23, 2023.
(60) Provisional application No. 63/486,474, filed on Feb. 23, 2023.
(60) Provisional application No. 63/539,809, filed on Sep. 21, 2023.
(60) Provisional application No. 63/539,809, filed on Sep. 21, 2023.

(51) Int. Cl. G01T 1/20; A61B 6/42

(52) U.S. Cl. CPC G01T 1/2002; A61B 6/4225

(57) ABSTRACT

An inventive imaging method includes: (a) causing a beam to travel from an emitter through an examination area for receipt at a detector; and (b) within the detector, (i) transforming the beam that is received into light, (ii) transforming the light into electrical signals representative of digital images corresponding to the examination area, including using a collector within the detector to collect the light as it passes to photosensitive areas of the collector without first passing through any wiring layer of the collector, and (iii) transmitting from the detector the data representative of digital images for display of the digital images to a user on a computing device. The detector includes wiring layers having stacked substrates attached together, each including one or more processing circuits for fast readout and dual native ISO. Other innovative imaging apparatus and methods include a stacked films-based image intensification and light recycling method.

### COPYRIGHT STATEMENT

Any new and original work of authorship in this document is subject to copyright protection under the copyright laws of the United States and other countries. Reproduction by anyone of this document as it appears in official governmental records is permitted, but otherwise all other copyright rights whatsoever are reserved.

### BACKGROUND

The present inventions generally relate to imaging apparatus and methods and, in particular, one invention relates to imaging apparatus and methods utilizing wiring layers having stacked substrates with processors that provide fast readout speeds and low-noise multiple signal amplification, and another invention relates to imaging apparatus and methods utilizing stacked films-based image intensification and light recycling. Preferred embodiments relate to such apparatus and methods used in the context of dental and medical X-ray imaging modalities.

The demand is still increasing for commercial, cinema, and scientific cameras, as well as for smartphones, having high speed, low noise, and ultra-high-definition (UHD) CMOS image sensors with resolutions beyond 4K. High speed is desirable for slow-motion applications and for reducing rolling shutter distortion for moving objects and subjects. Low noise is desirable for capturing a clear image in low-light conditions.

New advances in digital cameras and smartphones now allow for faster readout speeds thanks to the introduction of the stacked back-illuminated CMOS sensor, as shown in U.S. Pat. Nos. 5,773,379 and 8,773,562. Through this approach, digital cameras and smartphones can offer faster frames per second while minimizing the blur effect from moving objects and subjects in a still picture. To achieve this further, all components are positioned in a similar arrangement, but the design stacks the image signal processor, circuits and its ultra-fast dynamic random-access memory (DRAM) in additional layers into the same silicon instead of laterally or at the periphery. This allows for higher bandwidth, lower delays and, in the end, faster data transfer. In addition, low noise in low light conditions has been improved thanks to the incorporation of dual native ISO conversion gain to commercial digital cameras and smartphones, as shown in U.S. Pat. Nos. 7,075,049 and 8,648,287. “ISO” is a term that was originally used in film cameras and derived from the International Organization for Standardization (ISO), the German Standards Organization (DIN), and the American Standards Association (ASA), and represents the sensitivity of film and digital cameras to light (or ISO speed). ISO divided the scale of film to be sensitive to different levels of light. At the typical digital camera setup in professional photography and videography, the lens aperture function, the shutter speed, the exposure value, and the ISO settings play a pivotal role for image acquisition. The native ISO is the point where the sensor achieves its best signal-to-noise ratio (SNR) which is the point where the noise is the least and the camera can produce the best combination of color, contrast, dynamic range (DR), and other qualities. In the way commercial cameras use this dual native ISO approach, low-light images that otherwise would produce a higher noise in a higher ISO setting can extend their DR while reducing the signal noise that is produced. The camera can then read two native ISOs on the sensor instead of one. In these cameras, rather than one set of circuits adjusting the processing and transmitter to achieve a native ISO, there are two. Thus, while one native ISO may be 800, a second might be 3200.

In order to achieve a UHD image resolution in the commercial camera and smartphone field, a wider dynamic range (WDR) performance without SNR drop at the signal switching point is desired for improving sensing accuracy over the wide range of illumination conditions and ISO settings. In addition, suppression of motion blur is important when capturing moving objects and subjects. Several WDR technologies have been reported so far, including multiple exposure, dual photodiode (PD), dual conversion gain (DCG), lateral overflow integration capacitors (LOFICs), two-stage LOFICs, binary pixels, and combinations of the foregoing. The multiple exposure approach captures a few images with different exposure periods. The approach with dual PDs acquires an image with multiple PDs with different size or light sensitivity in a single exposure. The DCG approach changes conversion gain (CG) by controlling a switch connected to a capacitor and floating diffusion (FD) in a pixel during the horizontal blanking period. The LOFIC approach accumulates overflow-electrons from PD and FD capacitors and reads out signals with different sensitivity in a single exposure. This approach allows the independent design of CG and full well capacity (FWC), resulting in about 100 dB single exposure DR. The two-stage LOFICs allows increasing the DR while reducing sensor pixel size. In a similar approach, the binary pixels DR extension oversamples the incident light by using the ADC output at each sampling and resetting the pixel only if a threshold has been exceeded. Consequently, the maximum SNR on the high light side can be increased without decreasing sensitivity. In addition, single PD architecture is beneficial to maintain the optical performance when applying the image sensors to the various optical configurations.

Many imaging apparatus and methods are known. Current 2D and 3D dental X-ray imaging, dental fluoroscopy, digital tomosynthesis, photon-counting computed tomography, PET/SPECT scan devices, and general radiography, fluoroscopy, mammography, computed tomography and CBCT scan devices, all comprise front illuminated sensors. A traditional front-illuminated sensor is constructed in a fashion similar to the human eye, with a pixel gate and a digital matrix at the front and photosensitive areas at the back. This traditional orientation of the sensor places the active matrix of the digital image sensor—a matrix of individual picture elements—on its front surface and simplifies manufacturing. The matrix and its wiring, however, reflect some of the light, and thus the photosensitive areas can only receive the remainder of the incoming light; the reflection reduces the signal that is available to be captured resulting in a decrease in sensor quantum efficiency (QE). Consequently, front illuminated systems require increased radiation doses and large pixel sizes in order to gather enough light to produce an image. Patent references disclosing dental and medical imaging apparatus and methods following these principles include U.S. Pat. Nos. 4,057,727; 5,434,418; 5,834,782; 6,972,411; 7,016,461; 7,136,452; 7,197,109; 7,319,736; 7,322,746; 7,323,692; 7,336,763; 7,426,258; 7,563,026; 7,596,205; 7,608,834; 7,615,754; 7,629,587; 7,653,263; 8,378,310; and 9,180,215.

Current intraoral and extraoral digital dental X-ray systems comprise an X-ray source with a milliamperes (mA) setting ranging between 2.5 and 15 mA, a kilovolt peak (kVp) ranging between 60 and 120 kVp and exposure times ranging between 0.016 and 1.65 seconds for intraoral, and up to 20 seconds for extraoral. It is believed that intraoral dental systems did not allow frames per second until this feature was introduced through Uzbelger Feldman U.S. Pat. Nos. 6,543,936 and 8,430,563, and then for low-dose radiography through, for example, Uzbelger Feldman U.S. Pat. Nos. 10,849,586, 10,898,070 and 11,559,268, each of which are incorporated herein by reference. The apparatuses and methods disclosed therein exhibited increased sensor QE and fill factor for a lower dose over conventional devices, while allowing a pixel size reduction for an improved image resolution.

Thanks to the above efforts, the milliamperes (mA) range has been considered in attempts to reduce the radiation doses to which dental patients are exposed. The milliamperage is the units of the electrical current that is used to produce the radiation dose. The quantity, or number of X-rays emitted from the tube head, is controlled by the mA. With all other technical factors (e.g., kVp, time) held constant, patient radiation dose is directly proportional to the mA. A 50% reduction in mA results in a decrease in radiation dose by 50%. Radiation reduction in dentistry has been proposed through the introduction of low-dose static X-ray and fluoroscopy technology by minimizing the mA settings and the use of image intensification, as described in the Uzbelger Feldman '936 patent and '563 patent.

In particular, these recent breakthroughs in dose reduction made possible the production of low mA settings for dental use as disclosed in the Uzbelger Feldman '936 patent by using small image intensifiers between a detector's converter/plate and collector. Despite these efforts, a detector configuration using the image intensifier and collector was still too bulky to be used inside the mouth and not ergonomic for extraoral placement by the dentist while performing treatments on patients. Another disadvantage of intensifiers is image distortion originating from the projection of the X-ray image onto the curved input phosphor, and a smaller component corresponding to the mapping from the input phosphor to the output phosphor and the digital image matrix. In order to overcome these obstacles, a current attempt of on-chip image intensification for low milliamperes image capturing is known from the Uzbelger Feldman '563 patent by amplifying the electrical signals within the detector collecting area reducing a need for image intensifier coupling; however, in this approach no description of image collection at the photosensitive areas without passing through the wiring layer of the sensor is disclosed or suggested. As a result, this system would require a large pixel size ranging from 100 to 200 microns. In addition to the single wiring layer within the collector, there is only one circuit dedicated to electrical signal amplification.

A recent attempt described in the Uzbelger Feldman '070 patent is believed to lead to increased low-light collection efficiency in dental and medical radiology. In this regard, a microlens array is incorporated in the detector for collecting and focusing light, which would have otherwise fallen onto the non-sensitive areas of the collector. The use of a microlens array in imaging detectors between the converter/plate and the collector is believed to help increase image QE without compromising sensor size and image resolution. It is also seen to be advantageous in that there is no necessity to couple or attach additional bulky components, as a microlens array is very thin and fits well within a detector. The use of a microlens array also contributes to system pixel size reduction for an improved spatial resolution image.

Additional efforts to increase the light collection are disclosed in the Uzbelger Feldman '586 patent through a novel back-illuminated pixel architecture. Thinned back-illuminated CMOS sensor principles as disclosed in U.S. Pat. No. 4,266,334 are believed to have been invented around 1981. It is credited that the back-illuminated CMOS commercial adoption started when OmniVision developed their first sensors using the technique in 2007. The first widely used back-illuminated sensor was the OmniVision OV8810, which was announced in 2008, and comprised 8 megapixels and a 1.4 μm pixel size. The OV8810 was used in the HTC Droid Incredible and the HTC EVO 4G. Back-illuminated sensors currently are used in digital cameras and smartphones for capturing images in low light conditions, such as disclosed in U.S. Pat. No. 7,521,335. In addition, the use of back-illuminated CMOS sensors has been suggested for X-ray astronomy imaging purposes in U.S. Pat. No. 8,575,559; for X-ray diffraction detection and radiation monitoring in U.S. Pat. No. 8,421,007; for medical computed tomography in U.S. Pat. Nos. 10,761,219; 9,588,239; 8,288,733; 8,121,248; 7,869,559; 7,620,143; 7,455,454; and 6,426,991; and, for X-ray spectrometry purposes in U.S. Pat. No. 4,245,158.

Newer developments describe a stacked back-illuminated single-photon avalanche diode (SPAD) array sensor developed by Apple and disclosed in U.S. patent application publication 2018/0090536. Its main advantage over CMOS is low-light detection near-infrared (NIR) region. It is believed that the world's first commercial NIR stacked back-illuminated SPAD was implemented in the iPad 11 Pro in 2019. SPAD technologies have also been proposed for X-ray imaging detectors in U.S. Pat. No. 11,696,865 and in the Korean patent publication KR20230100090A of 2023 in which a stacked back-illuminated approach is not described.

Despite these advancements and a dose-reduction approach, data readout has space constraints laterally or at the sensor periphery while signal amplification can only be achieved through a single set of circuits within the system. This happens because a standard back-illuminated CMOS sensor has one layer in which the pixels and associated wiring circuitry are grouped together, and as a result, the primary task of collecting the light and processing is done within that layer, as disclosed in the Uzbelger Feldman '586 patent and '268 patent.

In view of the foregoing, additional improvements are believed to be desirable over the aforementioned imaging apparatus and systems, including all dental and medical X-ray imaging modalities such as intraoral and extraoral 2D and 3D dental imaging, digital tomosynthesis, photon-counting computed tomography, photon-counting radiography, PET/SPECT scans, general radiography, fluoroscopy, dental fluoroscopy, mammography, computed tomography and CBCT. In particular, it is believed that needs exist for a detector with higher megapixel counts that has improved readout speed, a detector that has lower-noise multiple signal amplification, and especially, a detector that has both improved readout speed and lower-noise multiple signal amplification. One or more aspects and features of the inventions are believed to address one or more such needs.

### SUMMARY

The inventive imaging apparatus and methods include many aspects and features. Moreover, while many aspects and features relate to, and are described in, the context of dentistry and medicine, the inventive imaging apparatus and methods are not limited to use only in such context, as will become apparent from the following summaries and detailed descriptions of aspects, features, and one or more embodiments of the inventive imaging apparatus and methods. Other fields that can benefit from the inventive imaging apparatus and methods include veterinary medicine, astronomy, industrial X-ray inspection, non-destructive testing, and airport security.

First inventive imaging apparatus and methods relate to imaging that utilizes stacked films-based image intensification and light recycling. In an aspect thereof, an imaging method comprises the step of: causing a primary light beam to travel from a primary light source through a stack of films; and at the stacked films: shielding X-rays and gamma radiation wavelengths; enhancing the beam into amplified light in different color spectrum wavelengths; intensifying the beam into a bright light; filtering and conducting the bright light onto photosensitive areas of a collector where the bright light is transformed into electrical signals; reflecting non-filtered light signals or secondary light into a rear mirror; and reflecting the secondary light from the rear mirror back to the stack of films to be conducted into the collector on a recycling approach. The primary light source preferably comprises an X-ray or gamma ray scintillator, a low-light body imaging area, a nanodots-based converter, or a combination thereof.

In a feature of this aspect, a component comprises the stacked films and is used in radiography, endoscopy cameras and capsules, catheters, intraoral cameras, intraoral 3D scanners, medical and dental robotics in providing healthcare services, including medical, dental, and veterinary fields for providing color and monochrome radiography and body imaging. Preferably, a detector of an imaging system comprises the stacked films.

In another aspect, a method comprises causing a primary light beam to travel from a source through a stack of films of a component of an imaging system, and throughout the films, performing a plurality of steps. The plurality of steps comprises: shielding X-rays and gamma radiation wavelengths; correcting a color wavelength of the primary light beam using filtering; enhancing the primary light beam into amplified light in different spectrum wavelengths; intensifying the amplified light into a bright light; conducting the bright light onto photosensitive areas of a collector, where it is transformed into electrical signals; reflecting non-filtered light signals or secondary light into a rear mirror; reflecting the secondary light from the rear mirror back to the stack of films toward the collector on a recycling approach; and transmitting digital data representative of digital images from the collector. The method further comprises performing the steps external to the collector comprising receiving digital data representative of digital images transmitted from the collector and processing the data representative of digital images for display of digital images to a user. In a feature, the method further comprises displaying the digital images to a user on a display.

In features, the primary light beam comprises an X-ray or gamma ray beam or a body imaging beam, and the shielding is performed using one or more radiation shielding layers located within the component. Preferably, a radiation shielding layer comprises a radiation-hardened fiber-optic plate, a radiation-hardened glass plate, a radiation-hardened microlens array, a radiation-hardened nano lens array, a radiation-hardened diffuser plate, a silica-based optical fibers radiation-hardened glass plate, a radiation-hardened nanoscale guiding light plate, or a combination thereof.

In another feature, the step of correcting of a color wavelength of the primary light beam is performed using one or more color filters located within the component. Preferably, a color filter comprises one or more of metal-organic frameworks, surface-mounted metal-organic frameworks, a color temperature orange (CTO) filter, a color temperature blue (CTB) filter, a color correction lighting gel, a NTSC filter, a red-green-blue (RGB) filter, a sRGB filter, an EXR filter array, a Quad RGB filter, a Nonacell filter, a cyan, magenta, yellow and white filter, a red, green, blue and white filter, a DCI P3 filter, a REC 2020 filter, an autochrome filter, a green filter, a red filter, a blue filter, a green and red filter, a green and blue filter, a red and blue filter, a green, red and blue mosaic filter, a green, red and blue vertically stacked filter, a CYGM (cyan, yellow, green magenta) filter, a RGBE (red, green, blue, emerald) filter, a RGBY (red, green, blue and yellow) filter, a magenta filter, a cyan filter, a cyan, magenta, blue filter, a yellow filter, an orange filter, panchromatic cells, color co-site sampling, X-trans filter, dichroic mirrors filter, triple-well filter, AR coating filter, broadband AR coating filter, UV coating filter, or UV-enhanced AR coating filter, a reflective filter, a diffractive filter, a refractive filter, a diffuser or a combination thereof.

In another feature, the step of enhancing the beam into amplified light is performed using one or more nanocrystal light boost films located within the component. In preferred embodiments, the nanocrystal light boost film is made from inorganic quantum dots, carbon-based quantum dots, perovskite quantum dots, or a combination thereof; the nanocrystal light boost film is made from core-type quantum dots, core-shell quantum dots, alloyed quantum dots, or a combination thereof; the nanocrystals are dispersed in a matrix layer between one or two light-transparent barrier layers; and the amount of nanocrystals and the crystal color ratio are based on the color or monochrome specifications of the application, the degree of light recycling and diffusion, the properties of the color filters and the film thickness.

In another feature, the step of intensifying the beam into a bright light is performed using one or more light intensity boost films located within the component. In preferred embodiments, the light intensity boost film is made from one of the groups of, organic materials, inorganic materials, or a combination thereof; the light intensity boost film comprises light focusing prisms, cones, 3-sided pyramids, 4-sided pyramids, triangles, spheres, rectangles, squares, rhomboids, octagons, hexagons, convex shape, concave shape, center-hollowed shapes, microlens-based structures, or a combination thereof; and two or more light intensity boost films can be stacked in a parallel direction, a perpendicular direction, a non-parallel direction with different angulations, a non-perpendicular direction with different angulations or a combination thereof.

In another feature, the step of conducting the bright light onto photosensitive areas of a collector is performed using one or more front reflective light conducting films located within the component. In preferred embodiments, the one or more front reflective light conducting films selectively filter and conduct the bright light into the photosensitive areas of the collector, reflect it back to the rear films'areas as a secondary light, or a combination thereof; and the one or more front reflective light conducting films comprise a linear polarizer, a circular polarizer, a reflective polarizer, a nano-grid reflective polarizer, a reflective microlens array, a dual light intensity boost film, a dual light intensity boost film with reflective polarizer, an anti-glare filter, a light-control film, or a combination thereof.

In another feature, the step of reflecting the non-filtered light signals or secondary light into a rear mirror is performed using one or more back mirror films located within the component. The one or more back mirror films preferably consist of a specular reflector, a white reflector, a transparent reflector film, or a combination thereof.

In another feature, a detector in an imaging apparatus comprises the component of the method. In preferred embodiments, an endoscopy camera or capsule comprises the component; a catheter comprises the component of the method; an intraoral camera comprises the component of the method; an intraoral 3D scanner comprises the component of the method; and medical and dental robotics comprises the component of the method.

In another feature, the collector comprises a complementary metal-oxide semiconductor (CMOS), a back-illuminated CMOS, a stacked back-illuminated CMOS, a charge-coupled device (CCD), a back-illuminated CCD, an active pixel sensor (APS), a photon counting detector, a back-illuminated photon counting detector, an amorphous silicon, an amorphous selenium, an N-type metal-oxide-semiconductor (NMOS), an APS thin film transistor (TFT), a single-crystalline silicon nanomembrane (Si NM), a Perovskite, halide Perovskite, lead halide Perovskite, single-crystalline Perovskite, or a combination thereof.

In another feature, the method further comprises displaying the digital images to a user on a display. Preferably, the step of displaying is performed locally or remotely on a wireless mobile computing device, a smartphone, a tablet, a laptop computer, a desktop computer, a virtual reality gadget, or a combination thereof.

In another aspect, an apparatus is configured to perform and performs one or more of the foregoing methods.

In another aspect, a system for use in radiography and body imaging in providing healthcare services comprises one or more of the following: a back mirror film located at the front or behind the light source; a radiation-shielded layer; a color filter; a nanocrystal light boost film; light intensity boost films and a front reflective light conducting film; a collector configured to transmit digital data representative of digital images based on light received at the collector; apparatus configured to receive the digital data representative of the digital images transmitted from the collector, process the data representative of digital images for display of the digital images to a user, and display the digital images to a user. Preferably, the digital images are displayed in 2D still images, 3D still images, 2D real-time video, 3D real-time video, 3D hologram or a combination thereof.

Second inventive imaging apparatus and methods relate to imaging apparatus and methods utilizing wiring layers having stacked substrates with processors that provide fast readout speeds and low-noise multiple signal amplification. In a first aspect thereof, an imaging method comprises the steps of: (a) causing a beam to travel from an emitter through an examination area for receipt at a detector; and (b) within the detector, (i) transforming the beam that is received into light, (ii) transforming the light into electrical signals representative of digital images corresponding to the examination area, including using a collector within the detector to collect the light as it passes to photosensitive areas of the collector without first passing through any wiring layer of the collector, and (iii) transmitting from the detector the data representative of digital images for display of the digital images to a user on a computing device. In this aspect, the detector comprises a plurality of wiring layers comprising stacked substrates attached together, each substrate comprising one or more processing circuits by which the detector is configured for fast readout speed and dual native ISO.

In a feature, the stacked substrates of the wiring layers are attached together by microbumps, direct bonding followed by Via-last through-silicon via (Via-last TSV), hybrid bonding (HB) technologies or a combination thereof.

In a feature, each of the stacked substrates of the wiring layers comprises a single wafer or a plurality of wafers. The wafers may be stitched, butted, or both.

In a feature, the detector comprises two or more wiring layers each comprising the substrate.

In a feature, the plurality of wiring layers is oriented behind one or more photosensitive areas in the direction of travel of the light within the detector.

In a feature, the stacked substrates of the wiring layers comprise one or more vertical pass gates; one or more floating diffusion nodes; one or more DRAM; WDR logic; readout circuitry; one or more analog-to-digital converters with a single or hybrid column counter and a scalable low-voltage signaling interface with an embedded clock (SLVS-EC) or a SLVS with a double-data-rate source-synchronous clock (DDR-SSC); column-parallel correlated multiple sampling (CMS) readout circuits with one or more output streams for controlling the switching of substreams at each frame; one or more digital-to-analog converters (DACs), and line buffers. For the stacked substrate of the SPAD approach, the wiring layers and pixel electronics could be a simple SPAD, a SPAD with a bit counter, a SPAD with a bit counter and a time-to-digital converter (TDC) or a combination thereof.

In a feature, the stacked substrates of the wiring layers comprise a plurality of analog-to-digital converters, and wherein the data is transmitted using the plurality of analog-to-digital converters for single or parallel multiple sampling readout and one or more output streams for controlling the switching of substreams at each frame. The computing device to which the data is transmitted preferably is a desktop or laptop computer, a wireless mobile computing device, a tablet, or smartphone, VR glasses, a headset, or a hologram projector. The method also preferably comprises the steps of receiving the fast readout speed and dual native gain transmitted data, processing the data, and displaying the digital images to a user on the computing device. The digital images displayed to a user may be 2D and 3D still images or real-time video.

In a feature, the method further comprises aiming the light to the photosensitive areas of the collector within the detector. The aiming of the light preferably is performed using a microlens array, an anti-glare filter, a light intensity boost film, a light control film or a color filter, a radiation-hardened or non-radiation-hardened fiber-optic plate or nano-optic plate, or a combination thereof.

In a feature, the stacked substrates of the wiring layers comprise a radiation resistant chip.

In a feature, the collector has a pixel size from 0.001 microns to 500 microns.

In a feature, transforming the beam into light is performed by one or more organic X-ray converters, an organic photoconductive film (OPF), an organic photodetector (OPD), inorganic X-ray converters, or a combination thereof, including Perovskite, halide Perovskite (inorganic, hybrid, organic-inorganic, 2/3D mixed dimensional and double Perovskite), lead halide Perovskite and single-crystalline Perovskite.

In a feature, the X-ray converter comprises a scintillator, a nanodots-based converter or a combination thereof.

In a feature, the X-ray converter comprises a nanodots-based converter that is made from inorganic quantum dots, carbon-based quantum dots, perovskite quantum dots, or a combination thereof.

In a feature, the X-ray converter comprises a solid, liquid, gas, or a combination thereof; the X-ray converter is coupled to the collector; the X-ray converter is coupled to a plate and the plate is coupled to the collector; and combinations thereof.

In a feature, the collector acts as an X-ray converter, such as in a direct radiography approach, photon counting, or a combination thereof.

In a feature, the beam caused to be emitted comprises a low-dose gamma ray or X-ray beam. The emitter preferably comprises one or more X-ray tubes, one or more gamma ray sources, or a combination thereof. With respect to the X-ray tubes, each X-ray tube preferably comprises a filament-based tube or a cold cathode-based tube such as a carbon nanotube having one or more focal spot sizes ranging from 0.001 microns to 3 mm. The nano focus and microfocus focal spot sizes will allow achievement of high resolution imaging at the cellular level for detecting early-stage disease.

In a feature, the detector comprises a lightproof housing within which components of the detector are enclosed. The lightproof housing preferably comprises a radiation-shielded back side that minimizes backscattered radiation.

In a feature, the detector comprises an intraoral or internal detector.

In a feature, the detector comprises an extraoral or external detector.

In a feature, the detector comprises an X-ray line detector with time-delay integration (TDI).

In a feature, the detector comprises a scalable tile detector.

In a feature, the detector comprises a flat panel detector.

In a feature, a housing of the detector comprises a coating layer that filters or reflects the light within the detector. The coating layer preferably is located on top of a converter.

In a feature, the method is used in dental digital radiography; low-dose dental digital radiography; dental fluoroscopy; dental panoramic scanning; dental cephalometric scanning; dental cone beam computed tomography (CBCT); linear tomography; digital tomosynthesis; dental X-ray stereoscopic spectroscopy; photon-counting computed tomography; photon-counting radiography, positron emission tomography (PET); single-photon emission computed tomography (SPECT); and general radiography, fluoroscopy, mammography and computed tomography.

In another aspect, an imaging method comprises the steps of: (a) causing a beam to travel from an emitter through an examination area for receipt at a detector; and (b) within the detector, (i) transforming the beam that is received into light, (ii) transforming the light into electrical signals representative of digital images corresponding to the examination area, including using a collector within the detector to collect the light as it passes to photosensitive areas of the collector without first passing through any wiring layer of the collector, and (iii) transmitting from the detector the data representative of digital images for display of the digital images to a user on a computing device. The collector comprises a stacked, back-illuminated sensor having two or more wiring layers by which the detector is configured for fast readout speed and dual native ISO.

In an aspect, an imaging method comprises: (a) causing a beam to travel from an emitter through an examination area for receipt at a detector; and (b) within the detector, (i) transforming the beam that is received into light, (ii) transforming the light into electrical signals representative of digital images corresponding to the examination area, including using a collector within the detector to collect the light as it passes to photosensitive areas of the collector without first passing through any wiring layer of the collector, and (iii) transmitting from the detector the data representative of digital images for display of the digital images to a user on a computing device. The detector comprises a plurality of wiring layers comprising stacked substrates attached together, each substrate comprising one or more processing circuits including amplifiers and one or more analog-to-digital converters, the amplifiers amplifying the electrical signals within the detector before being processed by the one or more analog-to-digital converters for single or parallel multiple sampling readout and one or more output streams for controlling the switching of substreams at each frame via multiple exposure, dual PD, two-stage LOFICs, binary pixels, or combinations thereof, to allow a dual native gain while minimizing SNR drop and maintaining a WDR.

In an aspect, a method of making a stacked, back-illuminated sensor comprises: (a) flipping a silicon wafer of a conventional detector so as to orient two or more wiring layers behind the one or more photosensitive areas of the wafer with respect to a direction of incident light to be received, and (b) thinning a side receiving the incident light so that the incident light strikes the one or more photosensitive areas of the wafer without passing through the two or more wiring layers of the wafer.

In an aspect, a low-dose radiation imaging apparatus comprises: (a) one or more emitters each configured to emit a low-dose gamma ray or X-ray beam through a patient examination area; and (b) one or more detectors each configured to receive the beam. Each detector comprises a housing containing: (i) a converter configured to transform the received beam into light; (ii) a collector that converts the light into electrical signals representative of digital images corresponding to the patient examination area, wherein the light that is collected passes to one or more photosensitive areas of the collector without passing through any wiring layer of the collector; and (iii) a transmitter that transmits from the detector data representative of digital images for display of the digital images to a user on a computing device. The detector comprises a plurality of wiring layers comprising stacked substrates attached together. The plurality of wiring layers comprises the transmitter and further comprises one or more vertical pass gates, one or more floating diffusion nodes, one or more DRAM, WDR logic, readout circuitry, one or more analog-to-digital converters with a single or hybrid column counter with a scalable low-voltage signaling interface with an embedded clock (SLVS-EC) or a SLVS with a double-data-rate source-synchronous clock (DDR-SSC), a column-parallel correlated multiple sampling (CMS) readout circuit with one or more output streams for controlling switching of substreams at each of a plurality of frames, a digital-to-analog converter (DAC), and line buffers. For the stacked substrate of the SPAD approach, the wiring layers and pixel electronics could be a simple SPAD, a SPAD with a bit counter, a SPAD with a bit counter and a time-to-digital converter (TDC), or a combination thereof.

As a result, a large number of frames per second and greater image acquisition are achieved with a reduction in radiation dosage, blur from moving objects and subjects during radiographic examinations or fluoroscopic procedures is minimized, seamless switching from video to still picture and vice versa, and slow-motion display are provided.

In a feature, the one or more emitters, the one or more detectors, or both, are configured to rotate around the examination area.

In a feature, one or more emitters, one or more detectors, or a combination thereof are mounted to a wall or ceiling by mechanical arms, C-arms, U-arms, or O-arm supports.

In a feature, one or more emitters, one or more detectors, or a combination thereof are attached to a fixed gantry.

In a feature, one or more emitters, one or more detectors, or both are handheld and portable.

In a feature, two or more emitters are configured to direct beams to the same detector.

In a feature, the emitter comprises one or more intraoral, extraoral, or external sources selected from the group of electromagnetic radiation, magnetic resonance, positron-emitting radionuclide, and a single-photon emission tracer.

In another aspect, a detector for use in dental and medical X-ray imaging modalities comprises a converter and a collector, the detector configured to transform an X-ray beam into light and then convert the light into electrical signals at one or more photosensitive areas of the collector without the light first passing through the two or more wiring layers of the collector.

In a feature of this aspect, the detector further comprises one or more sets of amplifier circuits that amplify the electrical signals within the detector before being processed by one or more analog-to-digital converters for single or parallel multiple sampling readout and one or more output streams for controlling the switching of substreams at each frame via multiple exposure, dual PD, two-stage LOFICs, binary pixels, or combinations thereof, in the two or more wiring layers to allow a dual native gain while minimizing SNR drop and maintaining a WDR.

It is believed that one or more aspects and features described above provide a faster readout allowing for a reduction in the pulsing width during static and pulsed X-ray imaging modalities for radiation dose reduction as well as seamlessly switching from video to still picture and vice versa and slow-motion display. It also is believed that one or more aspects and features described above provide a dual native gain allowing for a reduction in mA to an even lower dose mode thereby increasing patient safety without compromising signal-to-noise and image quality.

It is further believed that the faster readout speed will allow a detector with higher frame rates, which helps capture images when a patient moves with no blurring, thereby avoiding the need to redo imaging if the patient did not remain still. This is advantageous, especially for children. During fluoroscopy, high-speed procedures can be captured, like drilling on the tooth or bone without blurring. During CBCT, CT, or panoramic imaging, the gantry can be rotated faster around the patient thereby reducing exposure time and radiation dosage. This also allows slow-motion replay for teaching radiographic procedures to patients and students/residents. One or more aspects and features described herein are believed to provide processing light and electrical signals with an increased speed, even if the sensor includes higher megapixel counts, hence permitting reduction in the X-ray imaging detector pixel size for early-stage disease diagnosis accuracy.

It also is believed that the dual native ISO/gain allows an additional gain mode for capturing images in lower light conditions, in this case an even lower radiation dose. Thus, the mA can be reduced even more than conventional and low-noise X-ray images and video fluoroscopy can still be achieved. This can be used for screening procedures such as lung cancer radiographic imaging, CT, CBCT and mammography.

Additional features and embodiments that may utilize one or more of the aforementioned aspects are disclosed in the incorporated Uzbelger Feldman patents.

Still additional aspects, features, and embodiments are disclosed in U.S. provisional patent application 63/539,809 and U.S. nonprovisional patent application Ser. No. 18/380,530, from each of which priority is claimed and each of which is incorporated herein by reference.

In addition to the aforementioned aspects and features, it should be understood that the various possible combinations and subcombinations of the aspects and features of all of the inventive imaging apparatus and methods disclosed herein are contemplated and broadly encompassed within the scope of the invention. Moreover, claims in this or a divisional or continuing patent application or applications may be separately directed to any aspect, feature, or embodiment disclosed herein, or a combination thereof without requiring any other aspect, feature, or embodiment.

### BRIEF DESCRIPTION OF THE DRAWINGS

One or more preferred embodiments now will be described in detail with reference to the accompanying drawings.

FIG. 1 is a schematic illustration of imaging apparatus and methods in accordance with a preferred embodiment.

FIG. 1A is a schematic illustration of steps of a method performed by a detector in imaging apparatus and methods in accordance with a preferred embodiment.

FIG. 1B is a schematic illustration of steps of a method performed by a computing device in imaging apparatus and methods in accordance with a preferred embodiment.

FIG. 2 is a schematic illustration of imaging apparatus and methods in accordance with a preferred embodiment.

FIG. 3 is a schematic illustration of a detector in imaging apparatus and methods in accordance with a preferred embodiment.

FIG. 4 is a schematic illustration of a front illuminated architecture including a wiring layer and photosensitive layer.

FIG. 5 is a schematic illustration of a back-illuminated architecture including a wiring layer and photosensitive layer.

FIG. 6 is a schematic illustration of a back-illuminated architecture representative of two or more wiring layers in accordance with preferred embodiments.

FIG. 7 is a schematic illustration of an architecture for a detector in accordance with another preferred embodiment that utilizes stacked films-based image intensification and light recycling.

### DETAILED DESCRIPTION

As a preliminary matter, it will readily be understood by one having ordinary skill in the relevant art (“Ordinary Artisan”) that the inventive imaging apparatus and methods disclosed herein have broad utility and application. As should be understood, any embodiment may incorporate only one or a plurality of the above-disclosed aspects and may further incorporate only one or a plurality of the above-disclosed features. Furthermore, any embodiment discussed and identified as being “preferred” is considered to be part of a best mode contemplated for carrying out the inventive imaging apparatus and methods. Other embodiments also may be discussed for additional illustrative purposes in providing a full and enabling disclosure. As should be understood, any embodiment may incorporate only one or a plurality of the above-disclosed aspects and may further incorporate only one or a plurality of the above-disclosed features. Moreover, many embodiments, such as adaptations, variations, modifications, and equivalent arrangements, will be implicitly disclosed by the embodiments described herein and fall within the scope of the inventive imaging apparatus and methods described and enabled herein.

Accordingly, while the inventive imaging apparatus and methods are described herein in detail in relation to one or more embodiments, it is to be understood that this disclosure is illustrative and exemplary of the inventive imaging apparatus and methods and is made merely for the purposes of providing a full and enabling disclosure of the inventive imaging apparatus and methods. The detailed disclosure herein of one or more embodiments is not intended, and should not be construed, to limit the scope of patent protection afforded the inventive imaging apparatus and methods, which scope is to be defined by the claims and the equivalents thereof. It is not intended that the scope of patent protection afforded the inventive imaging apparatus and methods be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the inventive imaging apparatus and methods. Accordingly, it is intended that the scope of patent protection afforded the inventive imaging apparatus and methods is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Regarding applicability in the United States of 35 U.S.C. § 112(f) with regard to claim construction, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each in general denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers,” “a picnic basket having crackers without cheese,” and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Additionally, as used herein “low dose” in the context of X-rays and gamma rays is intended to mean an X-ray or gamma ray beam comprising a low milliamperes setting below 2.5 to 15 mA for digital imaging standards of dental intraoral and extraoral radiography, 50 mA for mammography, 100 mA for stationary X-ray units, and 50 mA for CT scans.

Referring now to the drawings, one or more preferred embodiments of inventive imaging apparatus and methods are next described. The following description of one or more preferred embodiments is merely exemplary in nature and is in no way intended to limit the scope of any claims.

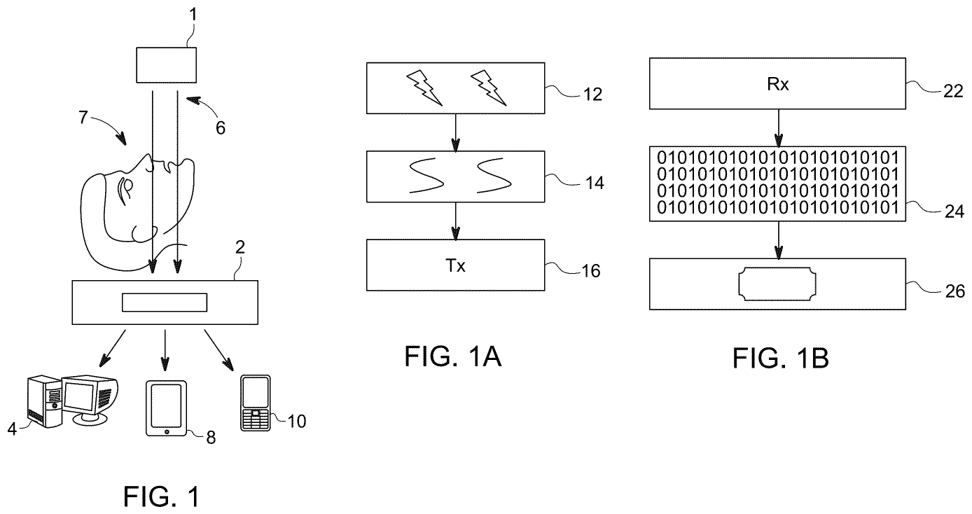

Turning now to FIG. 1, a schematic illustration of imaging apparatus and methods in accordance with a preferred embodiment of inventive imaging apparatus and methods are described. In this respect, a detector 2 may be used in all dental and medical X-ray imaging modalities, of which FIG. 1 is representative. As illustrated, an emitter 1 produces a beam 6 that travels through a patient examination area 7 and that is received at the detector 2.

The beam 6 emitted comprises gamma radiation or X-rays, both of which are referred to herein simply as X-rays. The emitter 1 preferably comprises one or more X-ray tubes, one or more gamma ray sources, or a combination thereof. With respect to the X-ray tubes, each X-ray tube preferably comprises a filament-based tube or a cold cathode-based tube such as a carbon nanotube having one or more focal spot sizes ranging from 0.001 microns to 3 mm. The nano focus and microfocus focal spot sizes will provide high resolution imaging at the cellular level for detecting early-stage disease. The detector 2 is shown in FIG. 1 as an extraoral or external detector. The detector 2 is used in X-ray imaging and may have a scintillator (indirect radiography) or may perform direct radiography or photon-counting with no scintillator.

The detector 2 preferably comprises a stacked, back-illuminated sensor that comprises a charge-coupled device (CCD), a complementary metal-oxide semiconductor (CMOS), an active pixel sensor (APS) CMOS, an N-type metal-oxide-semiconductor (NMOS), a pinned photodiode (PPD), avalanche photodiodes (APDs), a single-photon avalanche diode (SPAD) imager, an APS thin film transistor (TFT) or single-crystalline silicon nanomembrane (Si NM), crystalline selenium (c-Se), or a combination thereof.

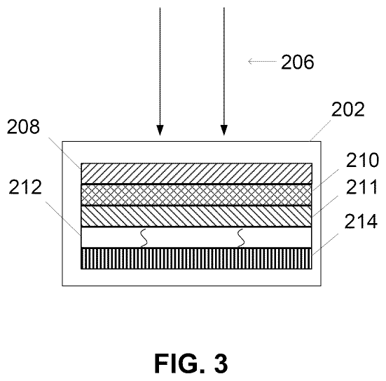

As schematically represented in FIG. 1A, certain preferred steps are performed within the detector 2, including a step 12 of transforming the beam 6 that is received at the detector 2 into light using a converter; a step 14 of capturing the light as it passes to a photosensitive layer of a collector within the detector, such as a photodiode layer, without first passing through any wiring layer, and to convert the light into electrical signals representative of digital images corresponding to the patient examination area 7, and further amplifying the electrical signals; and a step 16 of transmitting from the detector 2, based on the electrical signals, data representative of digital images for display of digital images to a user on a computing device. The detector 2 is further discussed in greater detail below with reference to FIG. 3.

The transforming of the beam into light may be performed by one or more organic X-ray converters, an organic photoconductive film (OPF), an organic photodetector (OPD), inorganic X-ray converters, or a combination thereof, including Perovskite, halide Perovskite (inorganic, hybrid, organic-inorganic, 2/3D mixed dimensional and double Perovskite), lead halide Perovskite and single-crystalline Perovskite.

In a feature, the X-ray converter comprises a scintillator, a nanodots-based converter, or a combination thereof.

In a feature, the X-ray converter comprises a scintillator, a nanodots-based converter that is made from one of the groups of inorganic quantum dots, carbon-based quantum dots, perovskite quantum dots, and combinations thereof.

Computing devices of users are schematically shown in FIG. 1 as including a desktop or laptop computer 4, a tablet 8, and a smartphone 10. The transmission of the data that is performed at step 16 within the detector preferably is received by such a computing device. Specifically, FIG. 1B schematically shows certain steps preferably performed within such a computing device, including the step 22 of receiving at such computing device the data transmitted from the detector 2 at step 16; the step 24 of processing, noise filtering, reconstructing, and enhancing the received data; and the step 26 of displaying the 2D or 3D digital images to a user on a display of the computing device. The computing device to which the data is transmitted preferably is a desktop or laptop computer, a wireless mobile computing device, a tablet, or smartphone, VR glasses, a headset, or a hologram projector. Exemplary such computing devices are represented in FIG. 1.

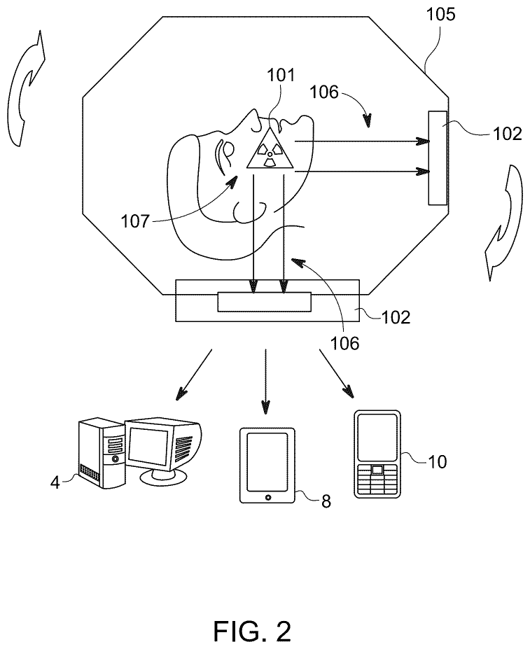

FIG. 2 is a schematic illustration of imaging apparatus and methods similar to the illustration of FIG. 1, but in which two or more detectors 102 mounted on a common support 105 for rotational movement around the patient examination area 107. The emitter 101 is shown in FIG. 2 as emitting radiation 106 and comprises an intraoral or internal source located inside of the patient that may include, for example, a miniature X-ray or gamma ray source, a positron-emitting radiotracer source, or a single-photon emission tracer.

It will be appreciated that while detector 2 and detectors 102 have been shown as extraoral or external detectors in the disclosed embodiments of FIGS. 1 and 2, intraoral or internal detectors can be used in other embodiments. Irrespective, in any such detector the light is collected at photosensitive areas without passing through any wiring layer. In accordance therewith, a beam 206 is converted into light and then focused, filtered, and collected at a photosensitive layer without passing through any wiring layers.

In particular, FIG. 3 is a schematic illustration of a detector used in imaging apparatus and methods in accordance with a preferred embodiment. The direction of travel of beam 206 comprising low-dose gamma rays or X-rays for detection by the detector is indicated by the arrows in FIG. 3. As shown in FIG. 3, the detector comprises a housing 202 in which is contained and enclosed one or more coating layers 208 including, for example, a grid, a refractive, a reflective, an anti-refractive or an antireflective layer; a converter 210, one or more focusing arrangements 211 including, for example, a radiation-hardened or non-radiation-hardened fiber-optic plate or nano-optic plate; a collector 212; and a transmitter 214. The one or more focusing arrangements 211 preferably comprises a microlens array, an anti-glare filter, a light intensity boost film, a light control film, a color filter, and combinations thereof.

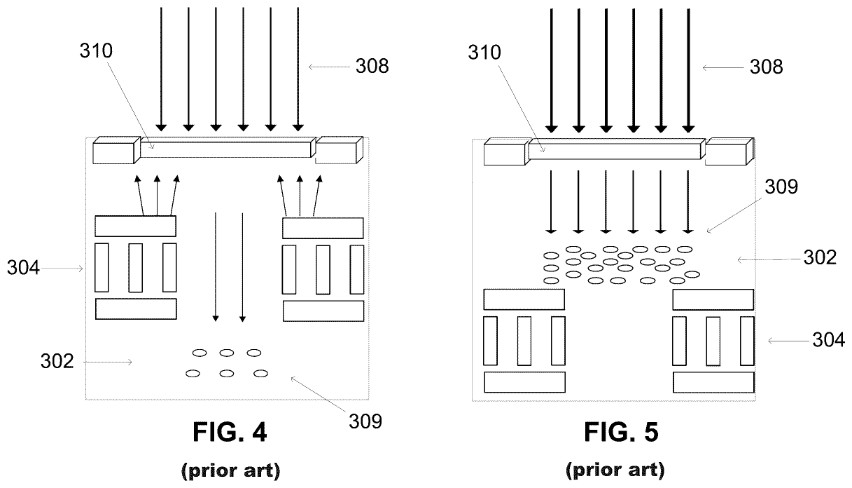

FIG. 4 schematically illustrates a front illuminated architecture of the prior art. This architecture of FIG. 4 includes a pixel gate 310, a wiring layer 304, and a photosensitive layer 302. The incident beam 308 encounters the pixel gate 310 and then the wiring layer 304 before encountering the photosensitive layer 302. Light is collected at area 309 behind wiring layer 304 relative to the direction of travel of light 308 in FIG. 4.

The collector 212 preferably has a pixel size from 0.001 microns to 500 microns.

FIG. 5 schematically illustrates a back-illuminated architecture of the prior art. Like the architecture of FIG. 4, this architecture of FIG. 5 includes a pixel gate 310, a wiring layer 304, and a photosensitive layer 302. Unlike the architecture of FIG. 4, the incident beam 308 encounters the pixel gate 310 and then the photosensitive layer 302 without passing through any wiring layer 304. Thus, light is collected at area 309 in front of wiring layer 304 relative to the direction of travel of light 308 in FIG. 5.

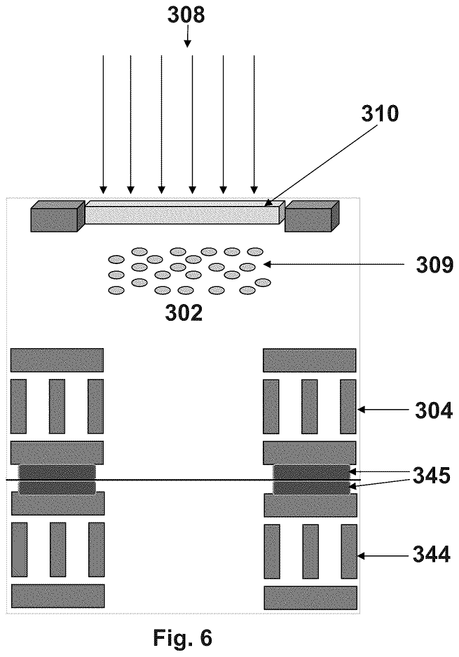

FIG. 6 schematically illustrates a stacked back-illuminated architecture in accordance with one or more aspects and features. Like the architecture of FIG. 5, the architecture of FIG. 6 includes a pixel gate 310, a wiring layer 304, and a photosensitive layer 302 at which light is collected in area 309. Furthermore, the incident beam 308 encounters the pixel gate 310 and then the photosensitive layer 302 without passing through any wiring layer 304. Thus, light is collected at area 309 in front of any wiring layer 304 relative to the direction of travel of light 308 in FIG. 6.

Unlike the architectures of FIG. 5 and FIG. 4, the architecture of FIG. 6 further includes a second wiring layer 344. The second wiring layer 344 is attached to the first wiring layer 304 through microbumps, direct bonding followed by Via-last through-silicon via (Via-last TSV), hybrid bonding (HB) technologies, or a combination thereof at 345. Additional wiring layers may be attached in a similar manner in accordance with additional embodiments. As shown in FIG. 6, the plurality of wiring layers is oriented behind the photosensitive areas in the direction of travel of the light within the detector.

In further regard to this, a detector in accordance with inventive imaging apparatus and methods comprises a plurality of wiring layers comprising stacked substrates attached together. Each substrate comprises one or more processing circuits. The additional wiring layers enable the detector to be configured for faster readout speeds and dual native ISO, which is believed to be a significant improvement over prior detectors in the context of dental and medical X-ray imaging modalities.

The stacked substrates of the wiring layers are attached together by microbumps, direct bonding followed by Via-last through-silicon via (Via-last TSV), hybrid bonding (HB) technologies, or a combination thereof. Each of the stacked substrates of the wiring layers comprises a single wafer or a plurality of wafers that may be stitched, butted, or both. As shown in FIG. 6, the detector comprises two wiring layers, each comprising a substrate with one or more processing circuits.

Preferably, the electrical signals into which the light is transformed are amplified by one or more sets of circuits before the signals are processed by one or more analog-to-digital converters for single or parallel multiple sampling readout and one or more output streams for controlling the switching of substreams at each frame via multiple exposure, dual PD, two-stage LOFICs, binary pixels, or combinations thereof in the two or more wiring layers to allow a dual native gain while minimizing SNR drop and maintaining a WDR.

In preferred embodiments the plurality of wiring layers comprises the transmitter as well as one or more vertical pass gates; one or more floating diffusion nodes; one or more DRAM; WDR logic; readout circuitry; one or more analog-to-digital converters with a single or hybrid column counter and a scalable low-voltage signaling interface with an embedded clock (SLVS-EC) or a SLVS with a double-data-rate source-synchronous clock (DDR-SSC); column-parallel correlated multiple sampling (CMS) readout circuits with one or more output streams for controlling the switching of substreams at each frame; one or more digital-to-analog converters (DACs), and line buffers. For the stacked substrate of the SPAD approach, the wiring layers and pixel electronics could be a simple SPAD, a SPAD with a bit counter, a SPAD with a bit counter and a time-to-digital converter (TDC), or a combination thereof. The data is transmitted using the plurality of analog-to-digital converters for single or parallel multiple sampling readout and one or more output streams for controlling the switching of substreams at each frame. The computing device to which the data is transmitted preferably is a desktop or laptop computer, a wireless mobile computing device, a tablet, or a smartphone. The computing device may further be VR glasses, a headset, or a hologram projector. The method also preferably comprises the steps of receiving the fast readout speed and dual native gain transmitted data, processing the data, and displaying the digital images to a user on the computing device via a field-programmable gate array (FPGA) interface card(s) and protocol(s). The digital images displayed to a user may be 2D and 3D still images or real-time video, and switching therebetween preferably is provided.

It is believed that one or more aspects and features described above provide a faster readout allowing for a reduction in the pulsing width during static and pulsed X-ray imaging modalities for radiation dose reduction as well as seamlessly switching from video to still picture and vice versa and slow-motion display. It also is believed that one or more aspects and features described above provide a dual native gain allowing for a reduction in mA to an even lower dose mode thereby increasing patient safety without compromising signal-to-noise and image quality.

It is further believed that the faster readout speed will allow a detector with higher frame rates, which helps capture images when a patient moves with no blurring, thereby avoiding the need to redo imaging if the patient did not remain still. This is advantageous, especially for children. During fluoroscopy, high-speed procedures can be captured, like drilling on the tooth or bone without blurring. During CBCT, CT, or panoramic imaging, the gantry can be rotated faster around the patient thereby reducing exposure time and radiation dosage. This also allows slow-motion replay for teaching radiographic procedures to patients and students/residents. It is believed that one or more aspects and features described herein provide processing light and electrical signals with an increased speed, even if the sensor includes higher megapixel counts, resulting in a reduction of the X-ray imaging detector pixel size for early-stage disease diagnosis accuracy.

It also is believed that the dual native ISO/gain allows an additional gain mode for capturing images in lower light conditions, in this case an even lower radiation dose. Thus, the mA can be reduced even more than conventional and low-noise X-ray images and video fluoroscopy can still be achieved. This can be used for screening procedures such as lung cancer radiographic imaging, CT, CBCT, and mammography.

Preferred methods may be used in dental digital radiography; low-dose dental digital radiography; dental fluoroscopy; dental panoramic scanning; dental cephalometric scanning; dental cone beam computed tomography (CBCT); linear tomography; digital tomosynthesis; dental X-ray stereoscopic spectroscopy; photon-counting computed tomography; photon-counting radiography, positron emission tomography (PET); single-photon emission computed tomography (SPECT); and general radiography, fluoroscopy, mammography and computed tomography.

Based on the foregoing description, it will be readily understood by the Ordinary Artisan that inventive imaging apparatus and methods are susceptible of broad utility and application. Many embodiments and adaptations of the inventive imaging apparatus and methods other than those specifically described herein, as well as many variations, modifications, and equivalent arrangements, will be apparent from or reasonably suggested and enabled herein.

For example, one or more of the emitters and detectors may be mounted to a wall or ceiling with appropriate supports or may be handheld and portable. Rotational support apparatus for the emitters and detectors also may be provided as disclosed, for example, in incorporated references, such as the Uzbelger Feldman '563 patent. Moreover, while FIG. 1 illustrates an external X-ray source being used with a detector, and FIG. 2 illustrates an internal X-ray source being used with two rotating detectors, one or more external X-ray sources may be used with one or more external detectors.

Additionally, it is contemplated that the stacked substrates of the wiring layers may comprise a radiation resistant chip. It also is contemplated that the X-ray converter may comprise a solid, liquid, gas, or a combination thereof; the X-ray converter is coupled to the collector; the X-ray converter is coupled to a plate and the plate is coupled to the collector; and combinations thereof.

It further is contemplated that the collector may act as an X-ray converter, such as in a direct radiography approach, photon counting, or a combination thereof.

It is also contemplated that the detector may comprise a lightproof housing within which components of the detector are enclosed. The lightproof housing preferably comprises a radiation-shielded back side that minimizes backscattered radiation.

In some embodiments, the detector may comprise an X-ray line detector with TDI, a scalable tile detector, and in other embodiments the detector may comprise a flat panel detector.

It also is contemplated that a housing of the detector may comprise a coating layer that filters or reflects the light within the detector, wherein the coating layer is located on top of a converter.

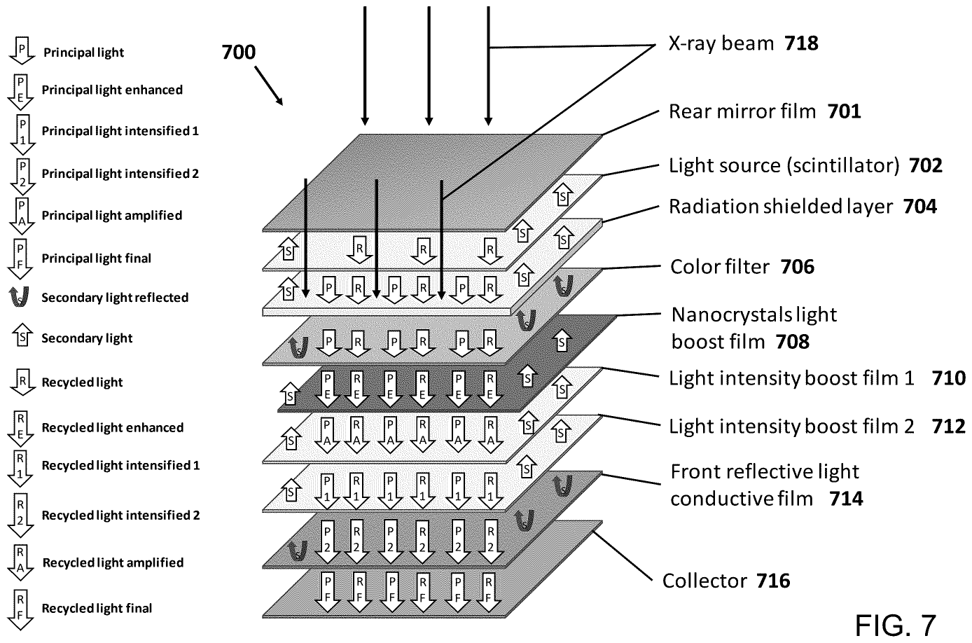

Still additional aspects, features, and embodiments of inventive imaging apparatus and methods are disclosed in provisional patent application 63/486,474, from which priority is claimed and which is incorporated herein by reference, and which are now described with reference to FIG. 7.

FIG. 7 schematically illustrates an architecture 700 for a component used in imaging in accordance with inventive imaging apparatus and methods. The architecture is configured to perform a stacked films-based image intensification and light recycling method. The method may be used in providing healthcare services in an internal component of X-ray detectors as well as in endoscopy cameras and capsules, catheters, intraoral cameras, intraoral 3D scanners and medical and dental robotics. In some preferred embodiments, the component is part of a detector used in radiography and low-light body imaging for medical, dental, and veterinary fields.

As shown, the architecture comprises a rear mirror film 701 located at the front or behind the light source 702, a radiation-shielded layer 704, one or more color filters 706, a nanocrystal light boost film 708, light intensity boost films 710,712, and a front reflective light conducting film 714. An X-ray beam 718 or body imaging primary light signals travel from the light source through a back mirror film, a radiation attenuation layer and a color correction filter and are then received at the nanocrystal light boost film. They are enhanced into the different color spectrum wavelengths. They are then amplified into a bright beam at the light intensity boost films and directed to the front film where they become selectively filtered and conducted into the photosensitive areas of the collector 716 or reflected. The non-filtered or secondary light signals reflected at the front film travel to the rear layer and then are mirrored back through the entire system toward the front film in a light recycling method until being filtered and conducted into the collector, thereby providing image intensification, and preventing light loss while increasing collector quantum efficiency and fill factor.

The stacked films-based image intensification and light recycling method comprises causing a primary light beam to travel from a primary light source through a stack of films; and at the stacked films: shielding X-rays and gamma radiation wavelengths; enhancing the beam into amplified light in different color spectrum wavelengths; intensifying the beam into a bright light; filtering and conducting the bright light onto photosensitive areas of a collector where the bright light is transformed into electrical signals; reflecting non-filtered light signals or secondary light into a rear mirror; and reflecting the secondary light from the rear mirror back to the stack of films to be conducted into the collector on a recycling approach.

It is believed that the system and method enable color and monochrome radiography and body imaging.

Accordingly, while inventive imaging apparatus and methods have been described herein in detail in relation to one or more preferred embodiments, it is to be understood that this disclosure is only illustrative and exemplary of the inventive imaging apparatus and methods and is made merely for the purpose of providing a full and enabling disclosure. The foregoing disclosure is not intended to be construed to limit the inventive imaging apparatus and methods or otherwise exclude any such other embodiments, adaptations, variations, modifications or equivalent arrangements, the inventive imaging apparatus and methods being limited only by the claims appended hereto and the equivalents thereof. Indeed, while preferred embodiments have been described in detail in the context of dentistry and medicine, the inventive imaging apparatus and methods are not limited to use only in such context, and other contexts of use include veterinary medicine, astronomy, industrial X-ray inspection, non-destructive testing, and airport security.

What is claimed is:

1. An imaging method, comprising the steps of:
(a) causing a primary light beam to travel from a primary light source through a stack of films of a component of an imaging system; and
at the films of the component, (b) shielding X-rays and gamma radiation wavelengths;
(c) enhancing the beam into amplified light in different color spectrum wavelengths;
(d) intensifying the beam into a bright light;
(e) filtering and conducting the bright light onto photosensitive areas of a collector where the bright light is transformed into electrical signals;
(f) reflecting non-filtered light signals or secondary light into a rear mirror; and
(g) reflecting the secondary light from the rear mirror back to the stack of films to be conducted into the collector on a recycling approach.

2. The imaging method of claim 1, wherein the primary light source comprises an X-ray or gamma ray scintillator, a nanodots-based converter, a low-light body imaging area, or a combination thereof.

3. The imaging method of claim 1, wherein a detector of the imaging system comprises the component.

4. The imaging method of claim 1, wherein the imaging system is used in radiography, endoscopy cameras and capsules, catheters, intraoral cameras, intraoral 3D scanners, medical and dental robotics in providing healthcare services, including medical, dental, and veterinary fields for providing color and monochrome radiography and body imaging.

5. An imaging method, comprising the steps of:
(a) causing a primary light beam to travel from a source through a stack of films of a component of an imaging system;
(b) within the component, performing the steps of:
(i) shielding X-rays and gamma radiation wavelengths;
(ii) correcting a color wavelength of the primary light beam;
(iii) enhancing the beam into amplified light in different spectrum wavelengths;
(iv) intensifying the beam into a bright light;
(v) conducting the bright light onto photosensitive areas of a collector where it is transformed into electrical signals;
(vi) reflecting the non-filtered light signals or secondary light into a rear mirror;
(vii) reflecting the secondary light from the rear mirror back to the stack of films toward the collector on a recycling approach, and
(viii) transmitting digital data representative of digital images from the collector; and
(c) external to the component, performing the steps of:
(i) receiving digital data representative of digital images transmitted from the collector, and
(ii) processing the data representative of digital images for display of digital images to a user.

6. The imaging method of claim 5, wherein the primary light beam of said step (a) comprises an X-ray or gamma ray beam or a body imaging beam.

7. The imaging method of claim 6, wherein said shielding is performed using one or more radiation shielding layers located within the component.

8. The imaging method of claim 7, wherein the radiation shielding layer comprises a radiation-hardened fiber-optic plate, a radiation-hardened glass plate, a radiation-hardened microlens array, a radiation-hardened nano lens array, a radiation-hardened diffuser plate, a silica-based optical fibers radiation-hardened glass plate, a radiation-hardened nanoscale guiding light plate, or a combination thereof.

9. The imaging method of claim 5, wherein correcting a color wavelength of the primary light beam is performed using one or more color filters located within the component.

10. The imaging method of claim 9, wherein the color filter comprises one or more of metal-organic frameworks, surface-mounted metal-organic frameworks, a color temperature orange (CTO) filter, a color temperature blue (CTB) filter, a color correction lighting gel, a NTSC filter, a red-green-blue (RGB) filter, a sRGB filter, an EXR filter array, a Quad RGB filter, a Nonacell filter, a cyan, magenta, yellow and white filter, a red, green, blue and white filter, a DCI P3 filter, a REC 2020 filter, an autochrome filter, a green filter, a red filter, a blue filter, a green and red filter, a green and blue filter, a red and blue filter, a green, red and blue mosaic filter, a green, red and blue vertically stacked filter, a CYGM (cyan, yellow, green magenta) filter, a RGBE (red, green, blue, emerald) filter, a RGBY (red, green, blue and yellow) filter, a magenta filter, a cyan filter, a cyan, magenta, blue filter, a yellow filter, an orange filter, panchromatic cells, color co-site sampling, X-trans filter, dichroic mirrors filter, triple-well filter, AR coating filter, broadband AR coating filter, UV coating filter, or UV-enhanced AR coating filter, a reflective filter, a diffractive filter, a refractive filter, a diffuser or a combination thereof.

11. The imaging method of claim 5, wherein enhancing the beam into amplified light is performed using one or more nanocrystal light boost films located within the component.

12. The imaging method of claim 11, wherein the nanocrystal light boost film is made from inorganic quantum dots, carbon-based quantum dots, perovskite quantum dots, or a combination thereof.

13. The imaging method of claim 11, wherein the nanocrystal light boost film is made from core-type quantum dots, core-shell quantum dots, alloyed quantum dots, or a combination thereof.

14. The imaging method of claim 11, wherein the nanocrystals are dispersed in a matrix layer between one or two light-transparent barrier layers.

15. The imaging method of claim 11, wherein the amount of nanocrystals and the crystal color ratio are based on the color or monochrome specifications of the application, the degree of light recycling and diffusion, the properties of the color filters and the film thickness.

16. The imaging method of claim 5, wherein intensifying the beam into a bright light is performed using one or more light intensity boost films located within the component.

17. The imaging method of claim 16, wherein the light intensity boost film comprises organic materials, inorganic materials, or a combination thereof.

18. The imaging method of claim 16, wherein the light intensity boost film comprises light focusing prisms, cones, 3-sided pyramids, 4-sided pyramids, triangles, spheres, rectangles, squares, rhomboids, octagons, hexagons, a convex shape, a concave shape, a center-hollowed shape, a microlens-based structure, or a combination thereof.

19. The imaging method of claim 16, wherein two or more light intensity boost films can be stacked in a parallel direction, a perpendicular direction, a non-parallel direction with different angulations, a non-perpendicular direction with different angulations or a combination thereof.

20. The imaging method of claim 5, wherein conducting the bright light onto photosensitive areas of a collector is performed using one or more front reflective light conducting films located within the component.

21. The imaging method of claim 20, wherein the one or more front reflective light conducting films selectively filter and conduct the bright light into the photosensitive areas of the collector, reflect it back to the rear films'areas as a secondary light, or a combination thereof.

22. The imaging method of claim 20, wherein the one or more front reflective light conducting films comprise a linear polarizer, a circular polarizer, a reflective polarizer, a nano-grid reflective polarizer, a reflective microlens array, a dual light intensity boost film, a dual light intensity boost film with reflective polarizer, an anti-glare filter, a light-control film, or a combination thereof.

23. The imaging method of claim 5, wherein reflecting the non-filtered light signals or secondary light into a rear mirror is performed using one or more back mirror films located within the component.

24. The imaging method of claim 23, wherein the one or more back mirror films consist of a specular reflector, a white reflector, a transparent reflector film, or a combination thereof.

25. The imaging method of claim 5, wherein a detector in an imaging apparatus comprises the component.

26. The imaging method of claim 5, wherein an endoscopy camera or capsule comprises the component.

27. The imaging method of claim 5, wherein a catheter comprises the component.

28. The imaging method of claim 5, wherein an intraoral camera comprises the component.

29. The imaging method of claim 5, wherein an intraoral 3D scanner comprises the component.

30. The imaging method of claim 5, wherein medical and dental robotics comprises the component.

31. The imaging method of claim 5, wherein the collector comprises a complementary metal-oxide semiconductor (CMOS), a back-illuminated CMOS, a stacked back-illuminated CMOS, a charge-coupled device (CCD), a back-illuminated CCD, an active pixel sensor (APS), a photon counting detector, a back-illuminated photon counting detector, an amorphous silicon, an amorphous selenium, an N-type metal-oxide-semiconductor (NMOS), an APS thin film transistor (TFT), a single-crystalline silicon nanomembrane (Si NM), a Perovskite, halide Perovskite, lead halide Perovskite, single-crystalline Perovskite, or a combination thereof.

32. The imaging method of claim 5, further comprising displaying the digital images to a user on a display.

33. The imaging method of claim 32, wherein displaying is performed locally or remotely on a wireless mobile computing device, a smartphone, a tablet, a laptop computer, a desktop computer, a virtual reality gadget, or a combination thereof.

34. An apparatus configured to perform the imaging method of any of claims 1-33.

35. A system for use in radiography and body imaging in providing healthcare services, comprising one or more of the following: a back mirror film located at the front or behind the light source; a radiation-shielded layer; a color filter; a nanocrystal light boost film; light intensity boost films and a front reflective light conducting film; a collector configured to transmit digital data representative of digital images based on light received at the collector; apparatus configured to receive the digital data representative of the digital images transmitted from the collector, process the data representative of digital images for display of the digital images to a user, and display the digital images to a user.

36. The system of claim 35, wherein the digital images are displayed as 2D still images, 3D still images, 2D real-time video, 3D real-time video, 3D hologram or a combination thereof.

37. An imaging method, comprising the steps of:
(a) causing a beam to travel from an emitter through an examination area for receipt at a detector; and
(b) within the detector,
(i) transforming the beam that is received into light,
(ii) transforming the light into electrical signals representative of digital images corresponding to the examination area, including using a collector within the detector to collect the light as it passes to photosensitive areas of the collector without first passing through any wiring layer of the collector, and
(iii) transmitting from the detector the data representative of digital images for display of the digital images to a user on a computing device;
(c) wherein the detector comprises a plurality of wiring layers comprising stacked substrates attached together, each substrate comprising one or more processing circuits by which the detector is configured for fast readout speed and dual native ISO.

38. The imaging method of claim 37, wherein the detector comprises a charge-coupled device (CCD), a complementary metal-oxide semiconductor (CMOS), an active pixel sensor (APS) CMOS, an N-type metal-oxide-semiconductor (NMOS), a pinned photodiode (PPD), avalanche photodiodes (APDs), a single-photon avalanche diode (SPAD) imager, an APS thin film transistor (TFT) or single-crystalline silicon nanomembrane (Si NM), crystalline selenium (c-Se), or a combination thereof.

39. The imaging method of claim 37, wherein the step of transforming the beam into light is performed by an organic X-ray converter, an organic photoconductive film (OPF), an organic photodetector (OPD), an inorganic X-ray converter, or a combination thereof, including Perovskite, halide Perovskite (inorganic, hybrid, organic-inorganic, 2/3D mixed dimensional and double Perovskite), lead halide Perovskite and single-crystalline Perovskite.

40. The imaging method of claim 37, wherein the step of transforming the beam into light is performed by an X-ray converter comprising a solid, liquid, gas, or a combination thereof.

41. The imaging method of claim 37, wherein the X-ray converter is coupled to the collector or the X-ray converter is coupled to a plate and the plate is coupled to the collector.

42. The imaging method of claim 37, wherein the stacked substrates of the wiring layers comprise a radiation resistant chip.

43. The imaging method of claim 37, wherein the collector has a pixel size from 0.001 microns to 500 microns.

44. The imaging method of claim 37, wherein the step of transforming the beam into light is performed by an X-ray converter comprising a scintillator, a nanodots-based converter, or a combination thereof.

45. The imaging method of claim 44, wherein the nanodots-based converter is made from one of the group of inorganic quantum dots, carbon-based quantum dots, perovskite quantum dots, or a combination thereof.

46. The imaging method of claim 37, wherein the collector acts as an X-ray converter, such as in a direct radiography approach, photon counting, or a combination thereof.

47. The imaging method of claim 37, wherein the imaging method is used in direct or indirect radiography.

48. The imaging method of claim 37, wherein the stacked substrates of the wiring layers are attached together by microbumps, direct bonding followed by Via-last through-silicon via (Via-last TSV) technologies, hybrid bonding (HB) technologies, or a combination thereof.

49. The imaging method of claim 37, wherein each of the stacked substrates of the wiring layers comprises a single wafer or a plurality of wafers that are stitched, butted, or both.

50. The imaging method of claim 37, wherein the plurality of wiring layers is oriented behind one or more photosensitive areas in the direction of travel of the light within the detector.

51. The imaging method of claim 37, wherein the stacked substrates of the wiring layers comprise one or more vertical pass gates; one or more floating diffusion nodes; one or more DRAM; WDR logic; readout circuitry; one or more analog-to-digital converters with a single or hybrid column counter and a scalable low-voltage signaling interface with an embedded clock (SLVS-EC) or a SLVS with a double-data-rate source-synchronous clock (DDR-SSC); column-parallel correlated multiple sampling (CMS) readout circuits with one or more output streams for controlling the switching of substreams at each frame; one or more digital-to-analog converters (DACs), and line buffers.

52. The imaging method of claim 37, wherein the stacked substrates of the wiring layers comprise single-photon avalanche diode (SPAD) arrays.

53. The imaging method of claim 52, wherein the SPAD arrays comprise a bit counter.

54. The imaging method of claim 53, wherein the SPAD arrays further comprise a time-to-digital converter (TDC).

55. The imaging method of claim 37, wherein the detector comprises one or more field-programmable gate array (FPGA) interface cards for transmitting from the detector the data representative of digital images for display of the digital images to a user on a computing device.

56. The imaging method of claim 37, wherein the stacked substrates of the wiring layers comprise a plurality of analog-to-digital converters, and wherein the data is transmitted using the plurality of analog-to-digital converters for single or parallel multiple sampling readout and one or more output streams for controlling the switching of substreams at each frame.

57. The imaging method of claim 56, wherein the computing device to which the data is transmitted is a desktop or laptop computer; a wireless mobile computing device; a tablet; a smartphone; VR glasses; a headset; or a hologram projector.

58. The imaging method of claim 56, further comprising the steps of receiving the fast readout speed and dual native gain transmitted data, processing the data, and displaying the digital images to a user on the computing device.

59. The imaging method of claim 58, wherein the digital images are displayed to a user as 2D and 3D still images or are displayed to a user as real-time video.

60. The imaging method of claim 37, further comprising aiming the light to the photosensitive areas of the collector within the detector.

61. The imaging method of claim 60, wherein the aiming of the light is performed using a radiation-hardened or non-radiation-hardened microlens array, a radiation-hardened or non-radiation-hardened nano lens array, an anti-glare filter, a light intensity boost film, a light control film or a color filter, a radiation-hardened or non-radiation-hardened fiber-optic plate or nano-optic plate, or a combination thereof.

62. An imaging method, comprising the steps of:
(a) causing a beam to travel from an emitter through an examination area for receipt at a detector; and
(b) within the detector,
(i) transforming the beam that is received into light,
(ii) transforming the light into electrical signals representative of digital images corresponding to the examination area, including using a collector within the detector to collect the light as it passes to photosensitive areas of the collector without first passing through any wiring layer of the collector, and
(iii) transmitting from the detector the data representative of digital images for display of the digital images to a user on a computing device via one or more field-programmable gate array (FPGA) interface cards and protocols;
(c) wherein the detector comprises a plurality of wiring layers comprising stacked substrates attached together, each substrate comprising one or more processing circuits including amplifiers and one or more analog-to-digital converters, the amplifiers amplifying the electrical signals within the detector before being processed by the one or more analog-to-digital converters for single or parallel multiple sampling readout and one or more output streams for controlling the switching of substreams at each frame via multiple exposure, dual PD, two-stage LOFICs, binary pixels, or combinations thereof, to allow a dual native gain while minimizing SNR drop and maintaining a WDR.

63. The imaging method of claim 62, wherein the detector comprises a charge-coupled device (CCD), a complementary metal-oxide semiconductor (CMOS), an active pixel sensor (APS) CMOS, an N-type metal-oxide-semiconductor (NMOS), a pinned photodiode (PPD), avalanche photodiodes (APDs), a single-photon avalanche diode (SPAD) imager, an APS thin film transistor (TFT) or single-crystalline silicon nanomembrane (Si NM), crystalline selenium (c-Se), or a combination thereof.

64. The imaging method of claim 62, wherein the stacked substrates of the wiring layers comprise single-photon avalanche diode (SPAD) arrays.

65. The imaging method of claim 64, wherein the SPAD arrays comprise a bit counter.

66. The imaging method of claim 64, wherein the SPAD arrays further comprise a time-to-digital converter (TDC).

67. An apparatus configured to perform the imaging method of any of claims 37-66.

68. A low-dose radiation imaging apparatus, comprising:
(a) one or more emitters configured to emit a low-dose gamma ray or X-ray beam through a patient examination area; and
(b) one or more detectors each configured to receive the low-dose gamma ray or X-ray beam;
(c) wherein each detector comprises a housing containing:
(i) a converter configured to transform the received beam into light;
(ii) a collector that converts the light into electrical signals representative of digital images corresponding to the patient examination area, wherein the light that is collected passes to one or more photosensitive areas of the collector without passing through any wiring layer of the collector;
(iii) a transmitter that transmits from the detector data representative of digital images for display of the digital images to a user on a computing device via one or more field-programmable gate array (FPGA) interface cards and protocols;
(iv) a plurality of wiring layers comprising stacked substrates attached together, each substrate comprising one or more processing circuits including amplifiers and one or more analog-to-digital converters, the amplifiers amplifying the electrical signals within the detector before the electrical signals are processed by the one or more analog-to-digital converters for single or parallel multiple sampling readout and one or more output streams for controlling switching of substreams at each frame via multiple exposure, dual PD, two-stage LOFICs, binary pixels, or combinations thereof, to allow a dual native gain while minimizing SNR drop and maintaining a WDR; and
(v) wherein the plurality of wiring layers comprises the transmitter and further comprises one or more vertical pass gates, one or more floating diffusion nodes, one or more DRAM, WDR logic, readout circuitry, one or more analog-to-digital converters with a single or hybrid column counter with a scalable low-voltage signaling interface with an embedded clock (SLVS-EC) or a SLVS with a double-data-rate source-synchronous clock (DDR-SSC), a column-parallel correlated multiple sampling (CMS) readout circuit with one or more output streams for controlling switching of substreams at each of a plurality of frames, a digital-to-analog converter (DAC), and line buffers, whereby a large number of frames per second and greater image acquisition are achieved with a reduction in radiation dosage, blur from moving objects and subjects during a radiographic examination or fluoroscopic procedure is minimized, switching from video to still picture and vice versa and slow-motion display are provided, and development of detectors with higher megapixel counts is enabled.

69. The imaging apparatus of claim 68, wherein the detector comprises a charge-coupled device (CCD), a complementary metal-oxide semiconductor (CMOS), an active pixel sensor (APS) CMOS, an N-type metal-oxide-semiconductor (NMOS), a pinned photodiode (PPD), avalanche photodiodes (APDs), a single-photon avalanche diode (SPAD) imager, an APS thin film transistor (TFT) or single-crystalline silicon nanomembrane (Si NM), crystalline selenium (c-Se), or a combination thereof.

70. The imaging apparatus of claim 68, wherein the stacked substrates of the wiring layers comprise single-photon avalanche diode (SPAD) arrays.

71. The imaging apparatus of claim 70, wherein the SPAD arrays comprise a bit counter.

72. The imaging apparatus of claim 70, wherein the SPAD arrays further comprise a time-to-digital converter (TDC).

73. The imaging apparatus of claim 68, wherein the one or more emitters each has one or more miniature gamma ray sources, positron-emitting radiotracer sources, or a single-photon emission tracer and is configured to emit a gamma ray beam through a patient examination area.

74. The imaging apparatus of claim 68, wherein the one or more emitters each has one or more focal spot sizes ranging from 0.001 microns to 3 mm and is configured to emit an X-ray beam through a patient examination area.

75. The imaging apparatus of claim 68, wherein the emitters are internal or intraoral emitters.

76. The imaging apparatus of claim 68, wherein the emitters are external or extraoral emitters.

77. The imaging apparatus of claim 76, wherein the emitters are stationary.

78. The imaging apparatus of claim 76, wherein the emitters are rotational.

79. The imaging apparatus of claim 76, wherein the emitters are handheld.

80. The imaging apparatus of claim 76, wherein the emitters are portable.
